(19) United States
(12) Patent Application Publication
(10) Pub. No.: US 2014/0030143 A1
(43) Pub. Date: Jan. 30, 2014

(54) Money Air Freshner

(71) Applicant: Alexander Siobhan, Tampa, FL (US)

(72) Inventor: Alexander Siobhan, Tampa, FL (US)

(21) Appl. No.: 13/560,919

(22) Filed: Jul. 27, 2012

(51) Int. Cl. A61L 9/00

(52) U.S. Cl. 422/4; 422/120

(57) ABSTRACT

A motivational air freshener that is scented, configured, and decorated to provide motivation to a user. The sight and scent of the motivational air freshener serves as an olfactory and visual reminder of monetary incentives. Some versions of the motivational air freshener may be configured to have the general appearance of a hundred dollar bill United States currency, which may include the scent of currency. Other versions my include a motivational symbols. The motivational air freshener includes a body portion, which may be sized in some scaled correspondence to a hundred dollar bill. The body portion may also include scent chemistry to disperse the fragrance of the currency. The body portion may also include a hole for receiving a chain which may be useful for hanging it inside a vehicle.

### CROSS-REFERENCE TO RELATED APPLICATIONS

The present Utility patent application claims priority benefit of the U.S. provisional application for patent Ser. No. 61/512,014 filed on Jul. 27, 2011 under 35 U.S.C. 119(e). The contents of this related provisional application are incorporated herein by reference for all purposes to the extent that such subject matter is not inconsistent herewith or limiting hereof.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to air fresheners. More particularly, one or more embodiments of the invention relate to motivational air fresheners.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that air fresheners are consumer products used in homes or commercial products used in restrooms, vehicles, closets, and refrigerators that typically emit a pleasant fragrance. Air fresheners introduce fragrance into the air of interior spaces either as droplets which transition to vapor or as the molecules of fragrance ingredients evaporating from a source. Fragrance diffuses into the air to mask other odors or to introduce a specific odor.

Typically, motivation is the psychological feature that arouses a person to action toward a desired goal and elicits, controls, and sustains certain goal directed behaviors. There are many approaches to motivation: physiological, behavioral, cognitive, and social. Motivation is the crucial element in setting and attaining goals.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a top view of both sides of an exemplary motivational air freshener scented, configured, and designed to resemble a hundred dollar bill United States currency, and joined with an exemplary support portion, in accordance with an embodiment of the present invention;

FIG. 2 illustrates a detailed perspective view of an exemplary motivational air freshener hanging from an exemplary mounting portion, in accordance with an embodiment of the present invention;

FIG. 3 illustrates a detailed perspective view of an exemplary user receiving motivation from an exemplary motivational air freshener, in accordance with an embodiment of the present invention; and

FIG. 4 illustrates a detailed perspective view of an exemplary motivational air freshener resting flat on an exemplary mounting portion, in accordance with an embodiment of the present invention.

Unless otherwise indicated illustrations in the figures are not necessarily drawn to scale.

### DETAILED DESCRIPTION OF SOME EMBODIMENTS

Embodiments of the present invention are best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

The present invention will now be described in detail with reference to embodiments thereof as illustrated in the accompanying drawings.

There are various types of motivational air fresheners that may be provided by embodiments of the present invention. In one embodiment of the present invention, the air freshener 100 may provide a motivational symbol 116 that is scented, configured, and decorated to provide motivation to a user 114. The motivation may include both positive and negative motivational fragrances, symbols, indicia, and configurations. Those skilled in the art, in light of the present teachings will recognize that the fragrance of the motivational air freshener may remain on the user's body and clothing long after the user leaves the area of the motivational air freshener. Similarly, the visual image of the motivational air freshener may remain in the user's memory, long after the user leaves the area of the motivational air freshener. In this manner, the user may remain motivated even when away from the motivational air freshener. In some embodiments, the sight and scent of the motivational air freshener may serve as a perpetual olfactory and visual reminder of monetary incentives. The motivational air freshener may include a unit of currency, including, without limitation, a hundred dollar bill United States currency, a thousand dollar bill, a million dollar bill, a large coin, a unit of gold, a unit of silver, and a foreign unit of currency. In any case, the monetary incentive includes the scent of its respective currency. In some embodiments, the sight and scent of the motivational air freshener serves as an olfactory and visual reminder of monetary incentives. However, in other embodiments, the motivational symbol may include, without limitation, a religious emblem, a photograph, a trophy for a sporting event, a political symbol, a battlefield scenario, an obese person, a financial institution replica, and text of a motivational phrase. In any of the aforementioned motivational symbols, the scent and visual image of the motivational air freshener correlate to the respective motivational symbol.

In one embodiment of the present invention, the motivational air freshener may include a body portion 102. The body portion may be configured as a rigid planar rectangle. In one embodiment, the planar rectangle may correlate in size and dimension to a United States currency bill, such as, without limitation, a hundred dollar bill. In some embodiments, the body portion may include a predetermined quantity of scent chemistry 104. The predetermined quantity of scent chemistry may include a fragrance of currency, or the appropriate fragrance for the respective motivational symbol. In some embodiments, the body portion may be fabricated from a material suitable for absorbing the predetermined quantity of scent chemistry to the point of saturation and then dispersing the resultant fragrance over time, including, without limitation, felt, cardboard, paper, sponge, cotton, and a nonwoven material. However, in another embodiment, the body portion may include a cavity configured to receive a liquid predetermined quantity of scent chemistry. The liquid may create sufficient vapor from the predetermined quantity of scent chemistry such that the fragrance disperses through apertures in the body portion. However, various means for dispersion of the fragrance may be employed by the body portion. In one embodiment of the present invention, the body portion may include an indicia 106. The indicia may provide the visual effect for motivation. In one embodiment of the present invention, the indicia may include, without limitation, text and graphics resembling a hundred dollar bill United States currency. The indicia may be depicted on both sides of a planar rectangle sized and dimensioned to replicate the hundred dollar bill. However, in other embodiments various currencies from a plethora of countries and times may be designed on the body portion.

In one embodiment of the present invention, the motivational air freshener may include a support portion 108. The support portion may allow the motivational air freshener join with a mounting portion 112. The support portion may include, without limitation, a chain, a string, an adhesive coated on the body portion, a suction cup, and a clip. In some embodiments, the body portion may include an aperture 110 for receiving the support portion. For example, without limitation, the chain may pass through the aperture and form a substantially annular support member. In this manner, the chain may hang on the mounting portion. The mounting portion may include, without limitation, an interior element of a vehicle, a rear view mirror, a cupboard, a closet, and a refrigerator.

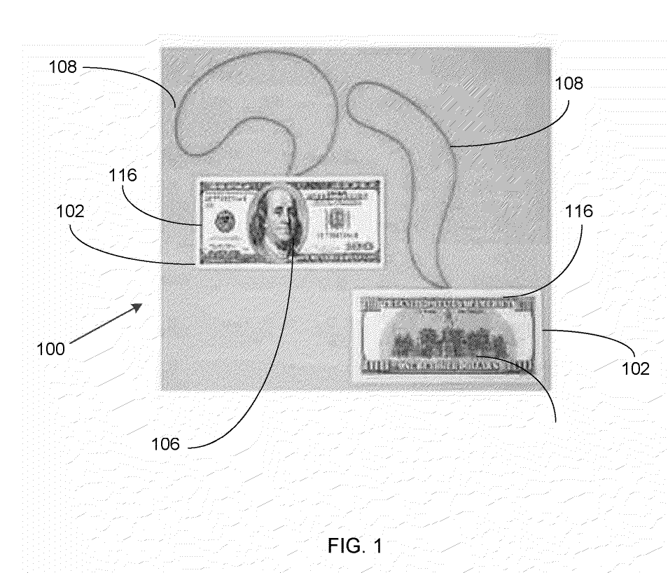

FIG. 1 illustrates a top view of both sides of an exemplary motivational air freshener scented, configured, and designed to resemble a hundred dollar bill United States currency, and joined with an exemplary support portion, in accordance with an embodiment of the present invention. In the present embodiment, the motivational air freshener may include a hundred dollar bill United States currency. To achieve this replica of the hundred dollar bill, the body portion may include a rigid planar rectangular configuration measuring 2.75″×4″. Those skilled in the art, in light of the present teachings will recognize that a currency with larger dimensions may be utilized to compensate for larger foreign bills, or a bill with smaller dimensions may be utilized for portability functions. In one embodiment of the present invention, each side of the body portion may include indicia resembling the hundred dollar bill United States currency. For example, without limitation, the color may include multiple shades of green, the depicted historical figure is Benjamin Franklin, and the seal of the United States Federal Reserve System is depicted, along with other features of the hundred dollar bill. However, in other embodiments, the currency may include novelty currency, whereby a comedian or other nonsensical figure is portrayed on the hundred dollar bill.

Those skilled in the art, in light of the present teachings will recognize that the predetermined quantity of scent chemistry may include, without limitations, oils, flowers, plants, and solvents. In some embodiments, the fragrance of United States currency may include, without limitation, a papery aroma with pink grapefruit, mandarin, freesia, passion fruit, Hawaiian wedding flower and melon; and a woody aroma of newly printed money with a fusion of fresh ocean breezes, bright citrus notes, rosemary, grass, and precious woods.

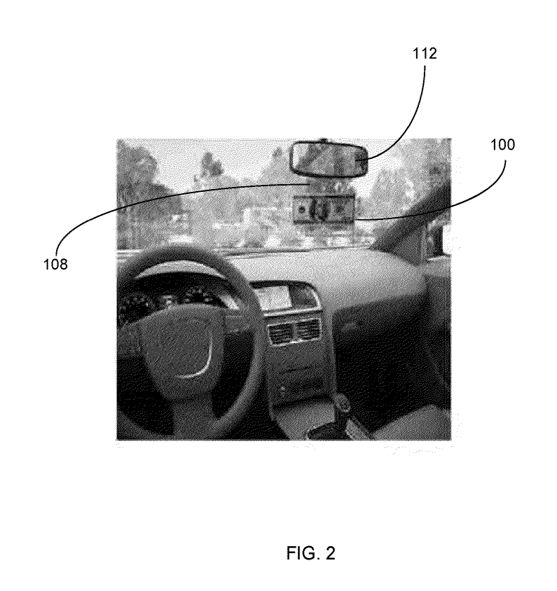

FIG. 2 illustrates a detailed perspective view of an exemplary motivational air freshener hanging from an exemplary mounting portion, in accordance with an embodiment of the present invention. In the present embodiment, the motivational air freshener may include a support portion. The support portion may allow the motivational air freshener join with a mounting portion. The support portion may include, without limitation, a chain, an elastic strap, a string, an adhesive coated on the body portion, a suction cup, a magnet, and a clip. Those skilled in the art, in light of the present teachings will recognize that the motivational air freshener may provide the greatest motivational influence when displayed prominently in proximity to the user. In some embodiments, the body portion may include an aperture for receiving the support portion. For example, without limitation, the chain may pass through the aperture and form a ring shaped support member operable to loop around the mounting portion. In this manner, the chain may hang on the mounting portion. The mounting portion may include, without limitation, an interior element of a vehicle, a rear view mirror, a cupboard, a closet, and a refrigerator. In one alternative embodiment, the support portion may hang from the user's neck, like a piece of jewelry. In this manner, the user will constantly be in proximity to the fragrance dispersed from the motivational air freshener.

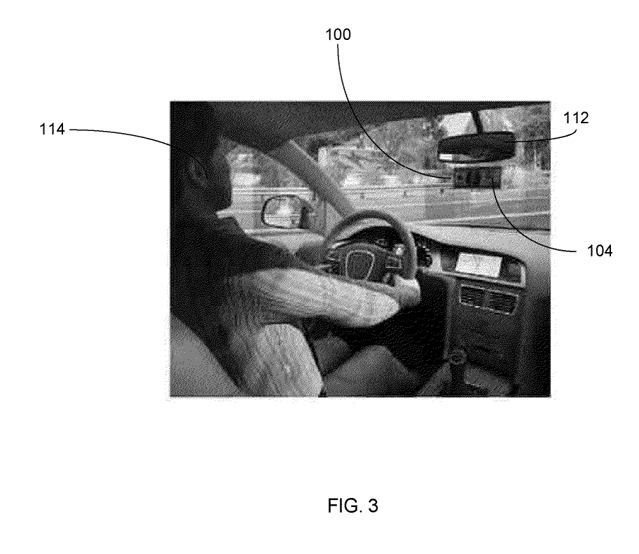

FIG. 3 illustrates a detailed perspective view of an exemplary user receiving motivation from an exemplary motivational air freshener, in accordance with an embodiment of the present invention. In the present embodiment, the motivational air freshener may provide an air freshener that is scented, configured, and decorated to provide motivation to the user. In one embodiment, the motivational air freshener may be positioned in sufficient proximity to the user, whereby the user constantly smells and sees the fragrance. Those skilled in the art, in light of the present teachings will recognize that the fragrance of the motivational air freshener may remain on the user's body and clothing long after the user leaves the area of the motivational air freshener. Similarly, the visual image of the motivational air freshener may remain in the user's memory, long after the user leaves the area of the motivational air freshener. In this manner, the user may remain motivated even when not in proximity to the motivational air freshener. The sight and scent of the motivational air freshener serves as an olfactory and visual reminder of the monetary incentives.

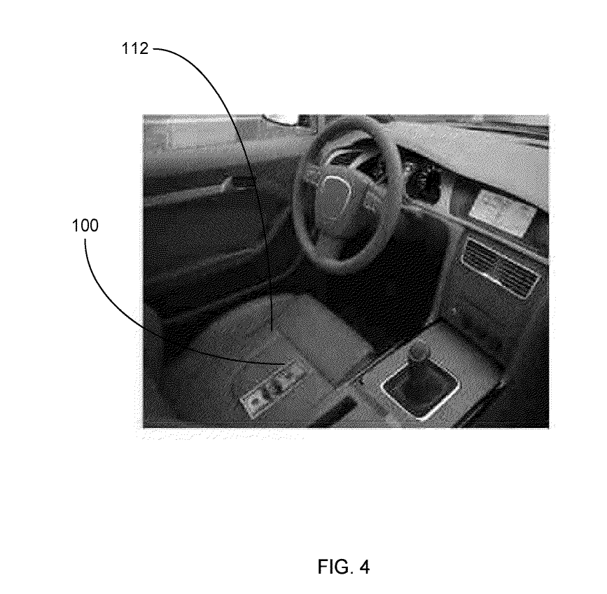

FIG. 4 illustrates a detailed perspective view of an exemplary motivational air freshener resting flat on an exemplary mounting portion, in accordance with an embodiment of the present invention. In the present embodiment, the motivational air freshener may position anywhere appropriate for dispersing the desired fragrance. In one embodiment, the motivational air freshener may rest flat against a flat surface. In yet another embodiment, the motivational air freshener may include an adhesive or a magnet for pressing against a refrigerator or a desk.

In one alternative embodiment, the body portion may include a cavity for storing the predetermined quantity of scent chemistry, and a fan for enhancing the dispersal of the fragrance. In yet another alternative embodiment, the motivational air freshener may include an audio device for emitting motivational music. In yet another alternative embodiment, the motivational air freshener may include an illumination portion for providing illumination.

All the features or embodiment components disclosed in this specification, including any accompanying abstract and drawings, unless expressly stated otherwise, may be replaced by alternative features or components serving the same, equivalent or similar purpose as known by those skilled in the art to achieve the same, equivalent, suitable, or similar results by such alternative feature(s) or component(s) providing a similar function by virtue of their having known suitable properties for the intended purpose. Thus, unless expressly stated otherwise, each feature disclosed is one example only of a generic series of equivalent, or suitable, or similar features known or knowable to those skilled in the art without requiring undue experimentation.

Having fully described at least one embodiment of the present invention, other equivalent or alternative methods of implementing motivational air fresheners resembling currency according to the present invention will be apparent to those skilled in the art. Various aspects of the invention have been described above by way of illustration, and the specific embodiments disclosed are not intended to limit the invention to the particular forms disclosed. The particular implementation of the motivational air fresheners resembling currency may vary depending upon the particular context or application. By way of example, and not limitation, the motivational air fresheners resembling currency described in the foregoing were principally directed to an air freshener having the fragrance, configuration, and indicia of a hundred dollar bill to provide monetary motivation implementations; however, similar techniques may instead be applied to air fresheners that smell like and visually resemble religious and sports competition symbols for motivating the user to pray and practice harder for a sporting competition, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

What is claimed is:

1. An air freshener comprising:
a body portion, said body portion being configured to resemble a motivational symbol, said body portion comprising a predetermined quantity of scent chemistry, said body portion further comprising an indicia.

2. The air freshener of claim 1, in which said body portion comprises a planar rectangle, said planar rectangle comprising two sides.

3. The air freshener of claim 2, in which said body portion comprises a currency.

4. The air freshener of claim 3, in which said body portion comprises a hundred dollar bill in United States currency.

5. The air freshener of claim 4, wherein said body portion disperses said predetermined quantity of scent chemistry.

6. The air freshener of claim 5, in which said predetermined quantity of scent chemistry comprises a fragrance of currency.

7. The air freshener of claim 6, in which said body portion comprises an aperture, said aperture being configured to receive a support portion, said support portion being configured to join with a mounting portion, said mounting portion being configured to support said body portion.

8. The air freshener of claim 7, in which said support portion comprises a chain.

9. The air freshener of claim 8, in which said mounting portion comprises an internal element of a vehicle.

10. The air freshener of claim 9, in which said indicia comprises a depiction of a hundred dollar bill in United States currency.

11. A method for motivating with a motivational air freshener comprising:
means for applying a predetermined quantity of scent chemistry on a body portion;
means for joining a support portion with a mounting portion;
means for displaying said motivational air freshener; and
means for receiving motivation from a fragrance and a visual image of said motivational air freshener.

12. An air freshener comprising:
a body portion, said body portion being configured to resemble a motivational symbol, said motivational symbol comprising a hundred dollar bill in United States currency, said body portion comprising a predetermined quantity of scent chemistry, said predetermined quantity of scent chemistry comprising a fragrance of currency, said body portion further comprising an indicia, said indicia comprising a decoration resembling said hundred dollar bill in United States currency, said body portion further comprising an aperture; and
a support member, said support member being configured to support said body portion, said support member being configured to pass through said aperture, said support member comprising a chain.
